(19) United States
(12) Patent Application Publication
(10) Pub. No.: US 2023/0157544 A1
(43) Pub. Date: May 25, 2023

(54) System and Methods for Remotely Monitoring an ICU Environment

(71) Applicant: QUANTAIRA, INC., New York, NY (US)

(72) Inventors: Todd J. Gross, New York, NY (US); Gengyuan Zhang, New York, NY (US); Noah Clay, Baldwinsville, NY (US); Ryan Saadi, Warren, NJ (US); Nathan lyer, Cape Coral, FL (US); Parmeswaren Kumar, Fort Lee, NJ (US); John F. Williams, Washington, DC (US)

(21) Appl. No.: 17/919,854

(22) PCT Filed: Apr. 16, 2021
(86) PCT No.: PCT/US21/27619

§ 371 Date: Oct. 19, 2022

Related U.S. Application Data
(60) Provisional application No. 63/117,795, filed on Nov. 24, 2020.
(60) Provisional application No. 63/083,508, filed on Sep. 25, 2020.
(60) Provisional application No. 63/012,717, filed on Apr. 20, 2020.

(51) Int. Cl. A61B 5/00; G06T 11/20; G06T 11/00

(52) U.S. Cl. CPC A61B 5/002; A61B 5/744; A61B 5/743; A61B 5/7475; G06T 11/206; G06T 11/001; A61B 2505/03

(57) ABSTRACT

Systems and methods for remotely monitoring hospital patients are provided. Medical devices may capture biometric data associated with a patient positioned in an ICU environment, and a remote display device positioned external to the ICU environment may display an indication of the health of the patient based on the captured biometric data. The indication of the health of the patient may include a graph mapping normalized health statuses associated with the patient over time. Each normalized health status may be based on normalizing a particular type of biometric data based on a medical protocol, so each data point of the graph includes an indication of a normalized health status associated with the patient at a given time. The indication of the health of the patient may include an animated three-dimensional that is dynamically updated to reflect biometric data associated with the patient captured at a time selected by a user.

### CROSS-REFERENCE TO RELATED APPLICATIONS

The present application claims priority to U.S. Provisional Patent Application No. 63/012,717, filed Apr. 20, 2020, entitled “System and Method Using Data Mining and Data Models to Manage and Control Ventilator Weaning Process;” U.S. Provisional Patent Application No. 63/083,508, filed Sep. 25, 2020, entitled “Systems and Methods for Remotely Monitoring an ICU Environment;” and U.S. Provisional Patent Application No. 63/117,795, filed Nov. 24, 2020, entitled “Systems and Methods for Remotely Monitoring an ICU Environment;” each of which are incorporated herein by reference in their entirety.

### FIELD OF THE DISCLOSURE

The present disclosure generally relates to systems and methods for remotely monitoring patients in an intensive care unit (ICU) environment, including systems and methods for managing and controlling the process of weaning a patient from a ventilator.

### BACKGROUND

Annually, in the United States (prior to COVID-19), more than 5.7M patients are admitted to an ICU. A range of 25-40% of patients are ventilated after admission and remain on ventilation for 15.5 days, on average. To this day, in the United States, approximately 3 episodes per 1000 hospitalizations involve mechanical ventilation. Senior citizens and patients in urban areas are highly likely to require mechanical ventilation. A study of hospital discharges in six states showed 52 out of 100 hospitalizations of the elderly required mechanical ventilation.

A 2005 study of hospital discharges in six states showed in-hospital mortality of ventilated patients to be 35%. At the time, the daily cost of mechanical ventilation averaged $1050 per day. In 2020, this average is estimated to jump to $2,000 per day, or about $31,000 per patient for ICU ventilator costs. In the United States, prior to COVID-19, the cost of ICU ventilation was approximately $39.7 B per year.

VAEs are Ventilator Associated Events that negatively impact patient outcomes—the most common of which are pneumonia and sepsis. Currently, 27% of critically ill patients develop pneumonia and 86% of these cases are Ventilator-Associated Pneumonias (VAP). VAP mortality rates have a reported range of 0 to 50%. Other common ventilator risks are air leaks, brain damage and sinus infection. Furthermore, there are approximately 200,000 instances per year of ALI (acute lung injury), including ventilator induced injuries, with an associated mortality rate of 40%. Typical causes of ALI are pneumonia, massive bleeding, a serious car accident or a life-threatening infection. The annual cost associated with ALI patients alone is $5.58 B per year. Patients in urban areas experienced additional challenges including a higher number of organ dysfunctions, tracheostomies and higher mortality when compared to their non-urban counterparts.

In the COVID-19 environment, as at other times, timely weaning from mechanical ventilation must be prioritized to minimize VAEs. That is, delivering the best possible care to patients on mechanical ventilation translates to weaning them from mechanical ventilation as soon as it is safely possible. However, discontinuing mechanical ventilation (extubation) continues to be one of the most challenging events in ICU management, and a significant portion of time spent on the ventilator (40%) is dedicated to weaning. Both premature and delayed extubation associated with adverse outcomes. The timing of extubation—which requires readiness of both the clinical team and patient—can vary day to day and throughout the same day. In addition, the method and risk of weaning varies among clinicians. Requirements for weaning from ventilation consist of both respiratory and non-respiratory parameters, all of which may play a critical role in weaning success and all of which the experienced intensivist considers at the time of weaning. For instance, requirements for weaning from ventilation include: adequate oxygen, adequate CO2 elimination, adequate respiratory muscular strength and ability to protect the airway are all examples of requirements for weaning from ventilation. Indicators of successful withdrawal of ventilation include a successful spontaneous breathing trial, minute ventilation, spirometry, PAO2/FlO2 ratio and rapid shallow breathing index.

Despite the use of various criteria for weaning, it is often difficult to determine the ideal time of extubation for each patient. Efforts have been made to solve the problem of weaning patients from mechanical ventilation. Kollef et al. showed that protocol-guided weaning of mechanical ventilation was safe and led to extubation more rapidly than physician-directed weaning. In a Multicenter trial, Esteban et al. compared different methods of weaning patients from mechanical ventilation. They concluded that a once-daily spontaneous breathing trial (SBT) led to extubation about three times more quickly than intermittent mandatory ventilation and about twice as quickly as pressure-support ventilation.

Weaning patients from mechanical ventilation is usually conducted in an empirical manner (physician driven). However, research has shown that protocol driven ventilator management and weaning leads to better outcomes (faster, fewer complications) than when weaning is directed/driven by physicians (slower, more complications). In the latter case, the number of ventilator adjustments is significantly greater.

Moreover, ventilator utilization protocols and weaning techniques vary from one health system to the next. The NIH has studied this non-uniformity and highlighted how hospitals/ICUs do not follow the same procedures surrounding ventilator management (i.e., protocol driven compared to physician driven). Decades of research on optimizing ventilator setting changes has shown how to use ventilators more safely and effectively; simple changes can decrease deaths from ALI by more than 20%. The American Thoracic Society describes how modifications to ventilator protocols has allowed patients with ALI to “survive illnesses which until recently, would have caused certain death.”

Furthermore, as mentioned, over 40% of total ventilator time may be consumed by the weaning process. Thus, weaning patients from ventilation constitutes a major portion of the workload in an intensive care unit. There are many clinical, logistical and practical challenges in an ICU environment associated with managing ventilated patients. Chief among these challenges are assessing the readiness of a patient to wean, daily spontaneous breathing trials (SBT), adherence to weaning protocols and constant analysis of ventilator settings alongside patient data to analyze the impact of adjustments on respiratory mechanics.

Additionally, in the COVID-19 environment, aside from ventilator shortages, there are further challenges with managing mechanically ventilated patients. Namely, high infection rates among healthcare workers, a shortage of personal protective equipment (PPE), a high rate of complications or mortality among patients and a shortage of clinical staff with critical care training. There is a 22% shortage of intensivists in 2020 in the United States. Consequently, when critical are physicians and nurses become infected, clinical teams from other areas of the hospital fill-in to treat their ICU patients. Many of these physicians and nurses have little ICU experience, particularly in managing ventilated patients.

Telemedicine in the ICU (e.g., eICU, tele-ICU, virtual ICU) provides remote monitoring of vital signs; for more detailed information, physicians typically consult the EMR remotely away from the ICU. In hospitals with tele-ICU systems, outcomes are very encouraging as patients were 26% more likely to survive the ICU, discharged from the ICU 20% faster, 16% more likely to survive hospitalization and be discharged as well as be discharged from the hospital 15% faster. However, most tele-ICU vendors do not provide ventilator settings to hospitals (owing to a myriad of equipment in use in any ICU).

### SUMMARY

In an aspect, systems and methods for remotely monitoring hospital patients are provided. One or more medical devices configured to capture one or more types of biometric data associated with a patient may be positioned in an intensive care unit (ICU) environment with the patient, and a remote display device positioned external to the ICU environment may be configured to display, via a user interface, an indication of the health of the patient based on the captured biometric data. For instance, the indication of the health of the patient may include a graph mapping one or more normalized health statuses associated with the patient over time. Each normalized health status may be based on normalizing a particular type of the captured biometric data based on a medical protocol, such that each data point of the graph includes an indication of a normalized health status associated with the patient at a given time. In some examples, the indication of the health of the patient may further include an animated three-dimensional model associated with the body of the patient, the animated three-dimensional model being dynamically updated to reflect biometric data associated with the patient captured at a time selected by a user.

In another aspect, a remote display device positioned external to an intensive care unit (ICU) environment is provided. The remote display device may be configured to display, via a user interface, an indication of the health of a patient positioned in the ICU environment based on biometric data captured by one or more medical devices positioned in the ICU environment. For instance, the indication may include a graph mapping one or more normalized health statuses associated with the patient over a period of time. Each normalized health status may be based on normalizing a particular type of the captured biometric data based on a medical protocol, such that each data point of the graph includes an indication of a normalized health status associated with the patient at a given time.

Furthermore, in still another aspect, a method for displaying an indication of health of a patient over time is provided. The method may include normalizing, by one or more processors, one or more types of biometric data associated with a patient based on a medical protocol to produce a normalized health status for each type of biometric data and generating, by the one or more processors, an indication of the health of the patient based on the biometric data. For instance, the indication may include a graph mapping one or more normalized health statuses associated with the patient over a period of time, such that each data point of the graph includes an indication of a normalized health status associated with the patient at a given time. The method may further include displaying, by the one or more processors, the generated indication of the health of the patient via a user interface display.

Additionally, in another aspect, a method for displaying an indication of health of a patient over time is provided. The method may include receiving, by one or more processors, one or more types of biometric data associated with a patient captured over a period of time and generating, by the one or more processors, an animated three-dimensional model associated with the body of the patient. One or more features of the animated three-dimensional model associated with the body of the patient may be dynamically updated as the one or more types of biometric data associated with the patient are change over the period of time. The method may further include displaying, by the one or more processors, the generated animated three-dimensional model associated with the body of the patient via a user interface display.

Moreover, in another aspect, a method for displaying an indication of health of a patient over time is provided. The method may include receiving, by one or more processors, one or more types of biometric data associated with a patient captured over a period of time; receiving, by the one or more processors, one or more text notes associated with the patient entered by a user over the period of time; storing, by the one or more processors, indications of times at which each of the one or more text notes were recorded; receiving, by the one or more processors, an indication of a term to be searched within the one or more text notes associated with the patient entered by the user; searching, by the one or more processors, the one or more text notes associated with the patient entered by the user for instances of the indicated term within the one or more text notes; displaying, by the one or more processors, an instance of the indicated term within the text notes via a user interface display; and displaying, by the one or more processors, a graphic based on the one or more types of biometric data associated with the patient captured at the time at which the text note containing the instance of the indicated term was entered.

Additionally, in another aspect, systems and methods for managing and controlling the process of weaning a patient from a ventilator are provided. A system for managing and controlling the process of weaning a patient from a ventilator may include ventilators positioned inside ventilator patient rooms, remote display devices associated with each ventilator positioned outside each ventilator patient room, and a computing device configured to analyze current patient information and current ventilator settings to determine metrics such as the current status of the ventilator patient, clinical recommendations regarding whether the ventilator patient is ready for weaning, clinical recommendations regarding timing and methodology of weaning steps likely to be successful for the ventilator patient, etc. The analysis may involve comparison to a ventilator weaning protocol and/or may involve the use of a predictive model. The computing device may provide the metrics (in some examples including graphics associated with the metrics) for display via the remote display devices positioned outside of the ventilator patient rooms.

### BRIEF DESCRIPTION OF THE DRAWINGS

FIG. 1 illustrates a block diagram of an example system for remotely monitoring patients in an intensive care unit (ICU) environment, including managing and controlling the process of weaning a patient from a ventilator.

FIGS. 1A-1H, 1J-1N, and 1P-1Z illustrate example user interface display screens that may be displayed via a remote display device used for remotely monitoring patients in an intensive care unit (ICU) environment, in accordance with some examples.

FIG. 2 illustrates a schematic diagram of variables that may be used in developing a predictive model for ventilator weaning.

FIG. 3 illustrates a flow diagram of an example method of managing and controlling the process of weaning a patient from a ventilator based on a ventilator weaning protocol.

FIG. 4 illustrates a flow diagram of an example method of managing and controlling the process of weaning a patient from a ventilator using a predictive model for ventilator weaning.

### DETAILED DESCRIPTION

The present disclosure provides a software and hardware platform that allows healthcare providers to easily observe biometrics from multiple medical devices attached to a patient in the intensive care unit (ICU). The platform aggregates readings and settings across a wide array of equipment such as ventilators, pulse oximeters, and other bedside monitors, and displays them on a single screen in real time through an application. These screens can be implemented via remote display devices, including tablets or other mobile computing devices that can be mounted outside of each patient's room in the ICU or at other appropriately secure locations (such as a nurses' station), or almost anywhere, subject to security protocols—or personal computers or smart phones. In short, this platform provides a window into the ICU from a safe distance outside—thus minimizing the need for health care personnel to physically enter the ICU for monitoring and recording data from the bedside monitors and ventilators.

Using this platform, healthcare professionals can see the biometrics of all patients in their care, whether the healthcare professional is onsite or offsite—in meeting rooms, at homes in hotels and so on. In addition, this platform allows providers to send customized instructions to ICU teams for display on the aforementioned tablets via a secure messaging channel. The mobile access feature is compatible with PCs, tablets and all smart devices.

There are many generations of bedside monitors and ventilators currently in use, ranging from those that are not integrated with Electronic Medical Record (EMR) systems and require manual charting, and those that stream all settings to the EMR. In some examples, this platform may be implemented via these existing bedside monitors and ventilators that have either been integrated with EMR systems or have published APIs which allow for real-time data capture, storage and display. Moreover, in some examples, this platform may be integrated into older generations of bedside monitors and ventilators, i.e., makes and models that do not readily support real-time data transmission. The data from such machines will be captured through a combination of methods including screen capture and available interfaces such as Recommended Standard 232 (RS-232). These devices will also need to be connectivity enabled, e.g., via an Ethernet cable or via Wi-Fi.

Generally speaking, all data capture, transmission, storage and display will be compliant with HIPAA regulations. In some examples, a multi-factor authentication (MFA), either using patient-specific fobs or other software mechanisms such as VIP Access/RSA, may be implemented in order to ensure the highest levels of cybersecurity.

Furthermore, the present disclosure provides a ventilator weaning platform that aids in clinical decision support through remote monitoring of ventilator settings and real time analysis of patient data with a focus on weaning from ventilation. The key thrust is reducing ventilator associated events (VAE) by weaning patients from mechanical ventilation in the most efficient and optimized manner. Generally speaking, a ventilator weaning platform accomplishes remote monitoring of ventilator settings via a screen that displays real time ventilator settings outside of each patient's room. To achieve this, data is streamed from a ventilator patient's electronic medical record (EMR) to, e.g., a cloud-based server, and then presented on the aforementioned screen.

In the course of accumulating patient data in the ICU, new ways of handling different disease conditions, new therapies, devices, treatments and technologies may be discovered. Identifying targets will bring about collaborations with pharmaceutical companies therapeutic indications may be recommended for a given population profile: usage, dosage, administration and comparator profiles. The healthcare team may control ventilators remotely or the ventilator weaning platform itself may control ventilators with or without human intervention. This will involve non-ICU applications, including but not limited to using the ventilator weaning platform in a sustaining mode for patients with ventilators at home, in skilled nursing facilities and long-term care facilities.

Automatic data collection can eliminate delays, improve charting efficiency, and reduce errors caused by incorrect data. In particular, electronic medical record (EMR) and ventilator data analysis with may improve patient outcomes by monitoring key factors such as: Fraction of Inspired Oxygen (FIO2), Positive End Respiratory Pressure (PEEP), Tidal Volume, Respiratory Rate, Peak Inspiratory Flow, Pressure Support, Peak Inspiratory Flor Rate, Low Minute Ventilation Alarm Limit, High Pressure Alarm Limit, High Respiratory Rate Alarm Limit, and Low Tidal Volume Alarm Limit. Analysis of this data can be used to update and customize automated ventilator protocols and thereby reduce the duration of mechanical ventilation. Gathering data from many thousands of patients and ventilators, examining successful and unsuccessful weaning outcomes, and comparing this information against reason for admission, demographics, co-morbidities, level of sedation, etc. has yielded a quantitative/predictive model that can be applied to a specific patient, thus allowing a ventilator weaning platform to be custom adjusted for each patient to optimize successful weaning and extubation.

For example, patient data (physical, medications, radiology, underlying disease conditions, etc.) along with ventilator settings may stream to the cloud, analysis may be performed, and data may streamed back to the displays outside of each patient's room and/or made available remotely to the clinical team. The power of this analysis is that the ventilator weaning platform will have access to data from many patients and will compare similar patients in its database to the exact patient in each room. Generally speaking, the ventilator platform aggregates data, performs a unique analysis/prediction and places that information in the hands of the care team, who will then make actual decisions regarding the ventilator (e.g., changing ventilator settings, initiating a spontaneous breathing trial, extubating, etc.). For example, a monitor outside of each patient room may display ventilator settings as well as a visual system (red/yellow/green background) highlighting each patient's trajectory on the ventilator as they progress in the ventilator weaning process.

Furthermore, in some examples, the ventilator weaning platform may include a clinical guidance tool rooted in this analysis and driven by protocol. The remote display device may also recommend/remind the treating healthcare professional (or team of healthcare professionals) of the need (when appropriate) for a spontaneous breathing trial (SBT).

Advantageously, the ventilator weaning platform makes ventilator information (including, e.g., ventilator settings information, recommendations, etc.) displayed on the screens of remote display device easily available to the clinical team on their phones and computers at any time and in any location. Beneficially, using this ventilator weaning platform, senior intensivists will minimize their chances of contracting COVID-19, efficiently guiding junior physicians and other members of the clinical team through ventilator management. Perhaps more importantly, one skilled intensivist will be able to monitor many ventilated patients simultaneously.

In particular, with a 22% shortage of intensivists in 2020 in the United States, there are multiple benefits associated with remote monitoring in the current crisis. Primarily, the ventilator weaning platform aims to keep critical specialists from becoming infected with COVID-19 (or other infectious diseases). When critical care physicians and nurses become infected, clinical teams from other areas of the hospital fill-in to treat their ICU patients. Many of these physicians and nurses have little ICU experience, particularly in managing ventilated patients. This ventilator weaning platform will be very helpful to an untrained or less trained workforce as more and more hospital staff are enlisted to help in ICUs. The remote display device will act not only as a guidance tool for mechanical ventilation, but will serve as an easy to interpret communications tool for patient status (e.g., preparing to wean, weaning, ready to extubate). Advantageously, with remote monitoring, senior critical care physicians are not required to enter each patient's room to evaluate ventilator settings as they can simply walk through the ICU hallways and view settings outside of each patient's room. Additionally, the use of PPE is minimized as each member of the clinical team is not required to don protective equipment to perform ventilator setting checks.

As an additional advantage, the ventilator weaning platform may be agnostic to the EMR system (e.g., Epic, Cerner) as well as the equipment being used in the ICU. Accordingly, all hospital systems may benefit from the analysis provided by the ventilator weaning platform, not just hospitals contracted to a particular vendor. For instance, eICU software that is only able to communicate with other certain vendors can underserve both patients and critical care teams. Moreover, the ventilator weaning platform will reinforce best practices/outcomes in one ICU and share with others; conversely, practices with less favorable outcomes will be highlighted for physician review.

Generally speaking, as discussed above, the analysis performed by the ventilator weaning platform involves accessing protected health information (PHI) from patients' EMR. To this end, the ventilator weaning platform employs best-in-class methods to ensure that that patient information, privacy, integrity and confidentiality data remain secure at all times. In particular, accessing PHI must incorporate the following security policies and procedures: authorization, authentication, availability, confidentiality, data integrity, and nonrepudiation (transaction records). Additionally, physical safeguards may include: use of encrypted storage or devices, restricting access to authorized personnel only, reserving copies and conducting data backups, maintaining emergency contingency protocols, and disposing outdated devices and data sources properly. For example, each remote display device may require an authentication process that links it with a given patient in a given room. In one example, disposable smart card that is unique to each patient may be inserted in each remote display device. The smart card may be programmed by hospital IT for each unique patient and, after insertion in each remote display device, the ventilator weaning platform will automatically authenticate the card with the patient's EMR and synchronize with the cloud.

Moreover, decisions that physicians make relative to each patent, their associated EMR data, and ventilator settings may be observed and recorded. In particular, this data may be collected, aggregated, and analyzed to identify parameters that lead to successful extubation. As the ventilator weaning platform aggregates more patient data, a seamless and virtuous feedback loop may be created, in which the ventilator weaning platform gains prediction accuracy and develops patient and disease specific guidance. Where there are no ventilator management protocols for certain diseases (e.g., new diseases such as COVID-19), the platform can analyze all patients and recommend standard treatment guidelines that can be evaluated by physicians. Additionally, although a ventilator weaning platform is discussed herein, similar platforms may be used to identify other correlations in critical care medicine beyond respiratory support.

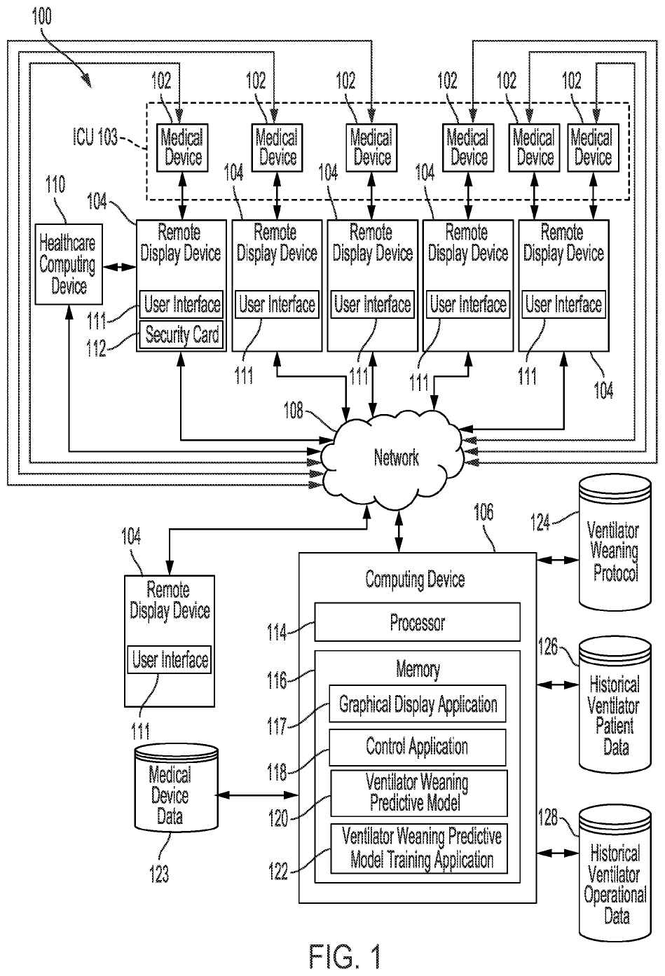

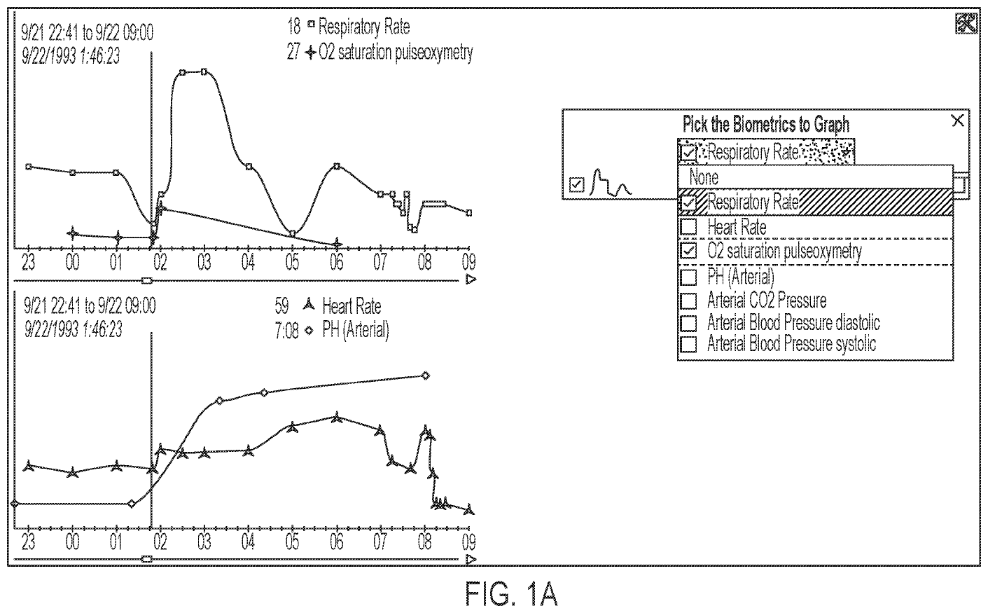

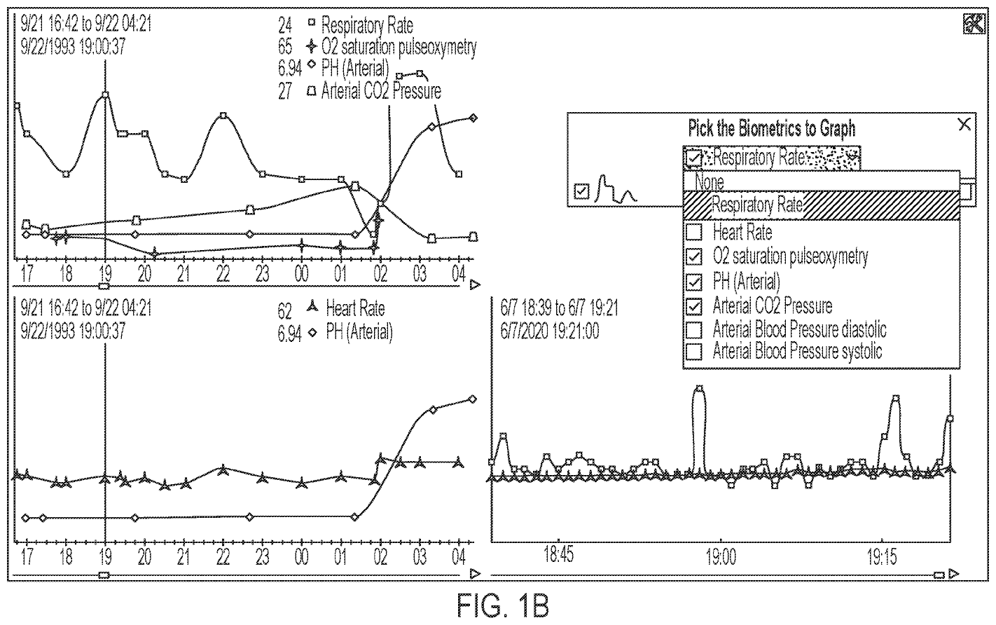

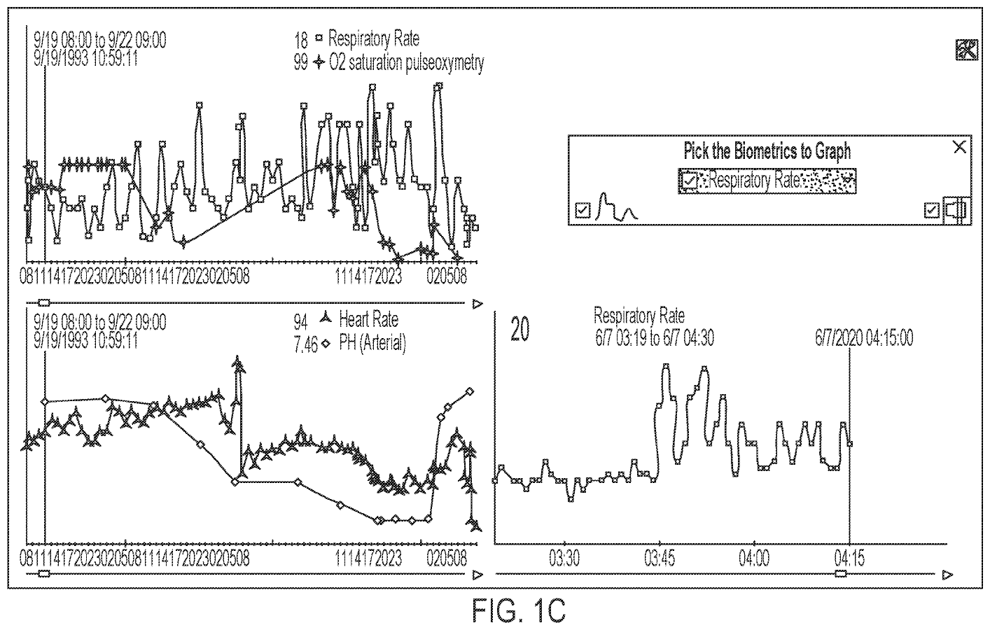

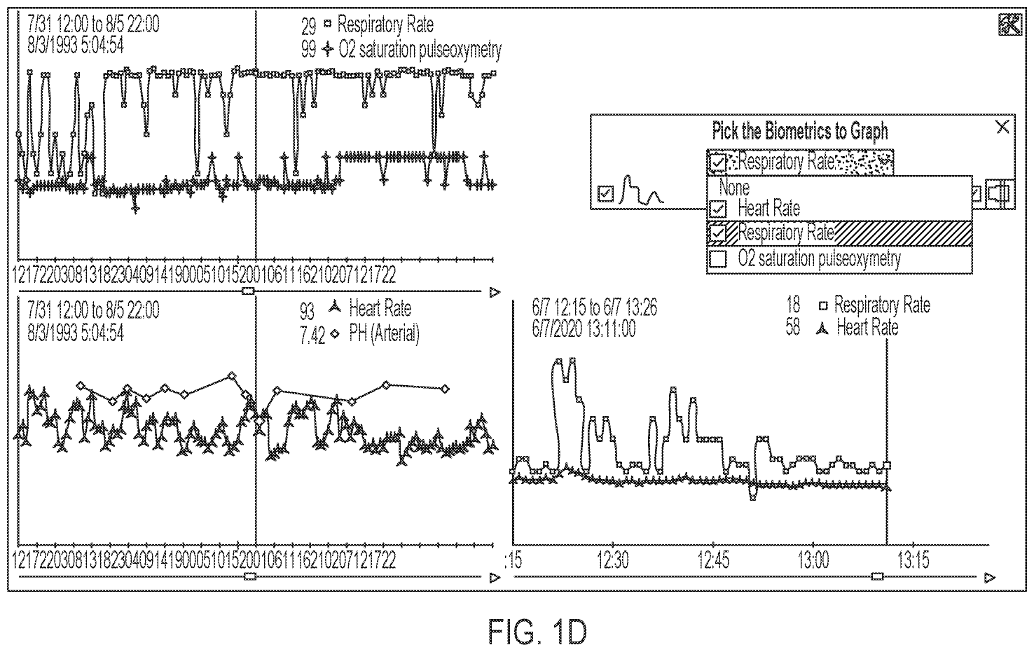

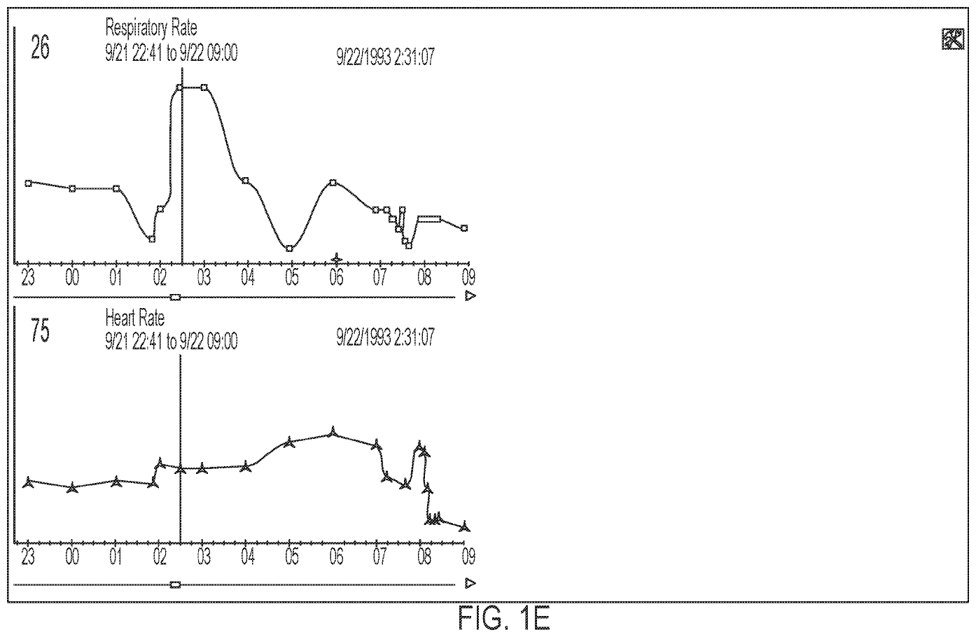

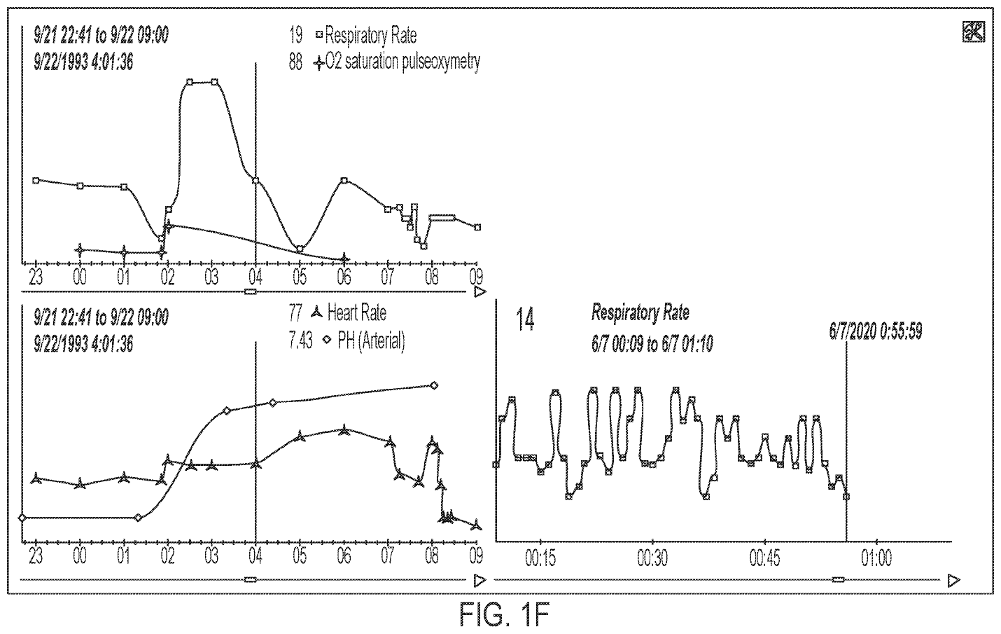

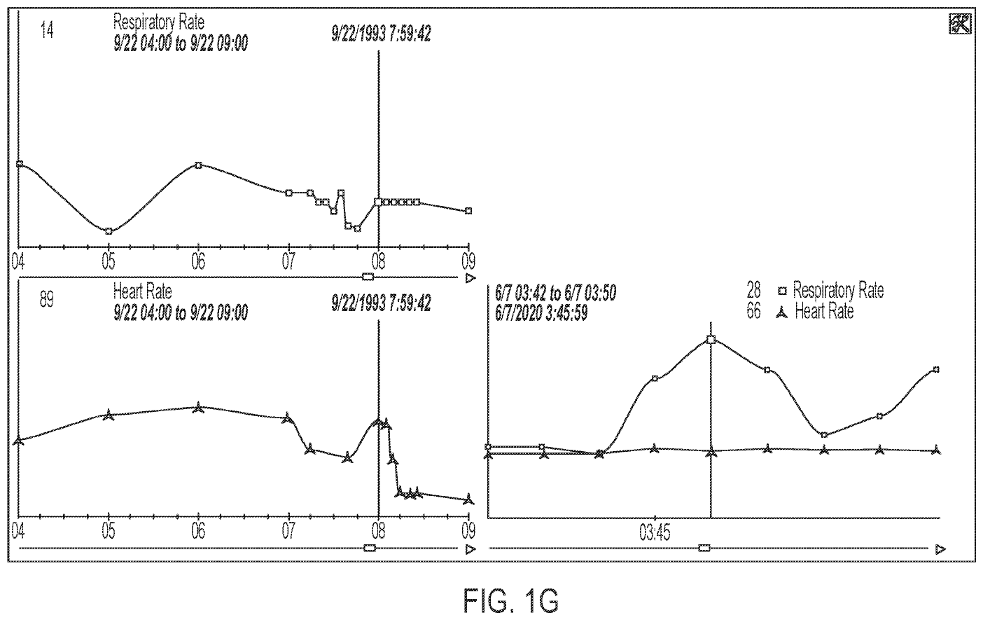

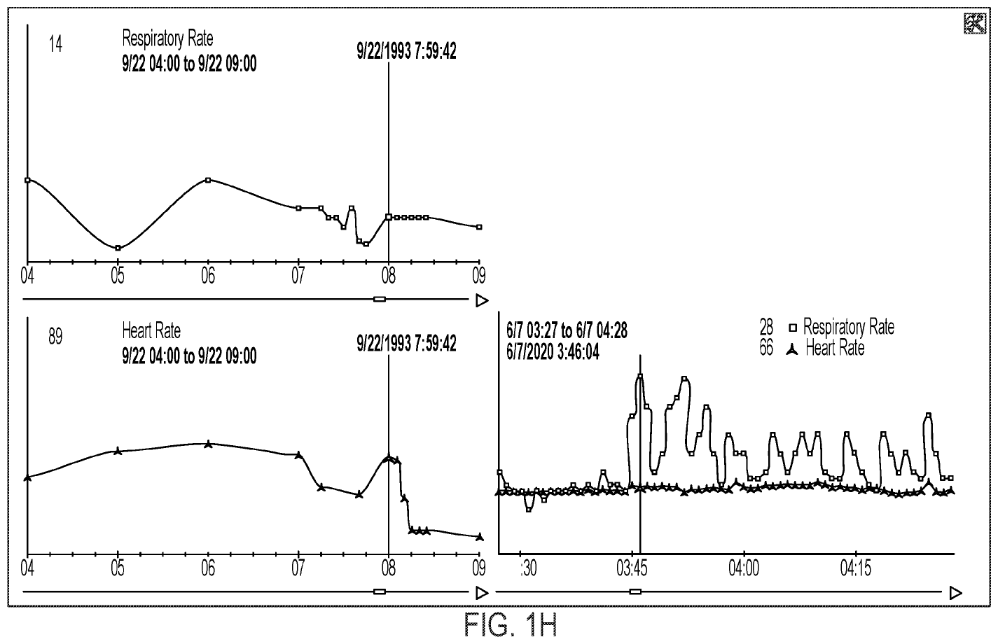

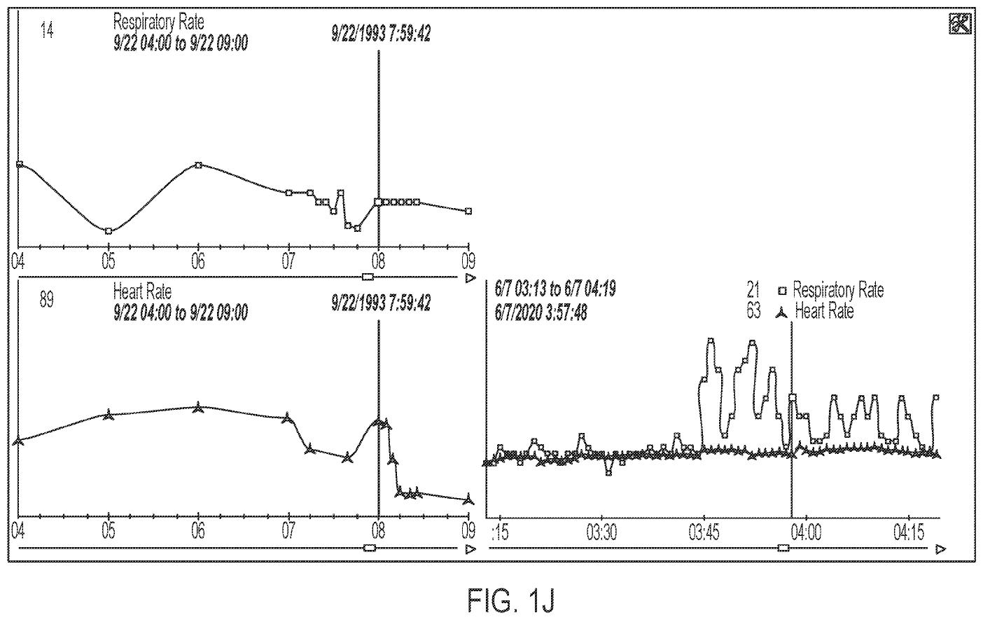

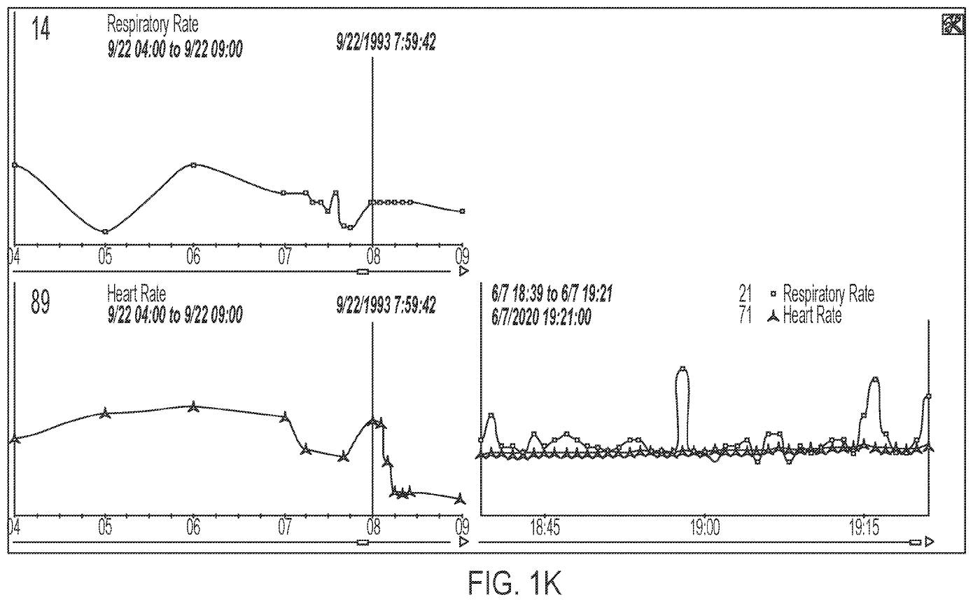

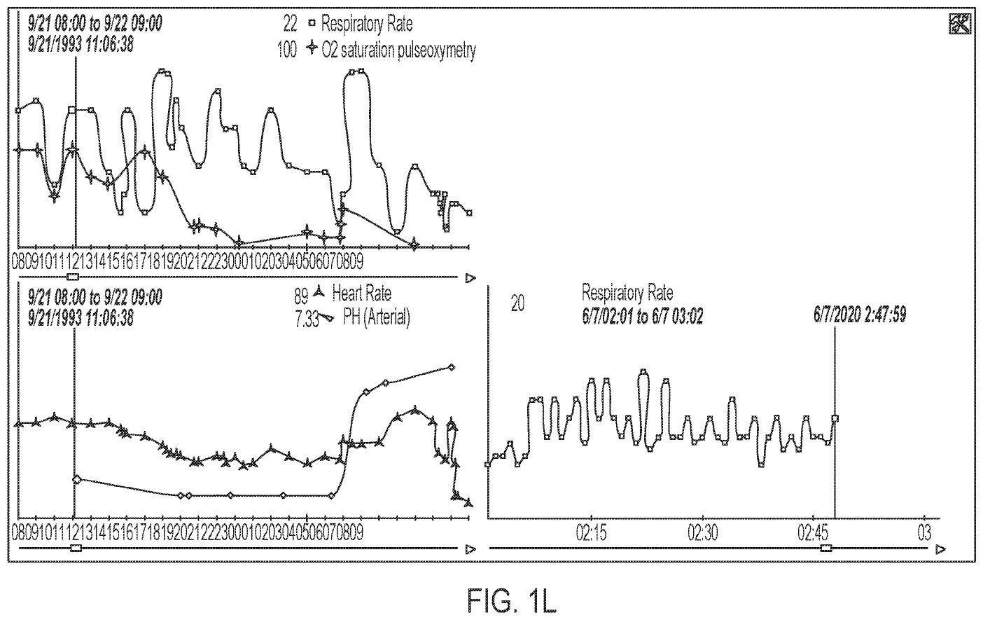

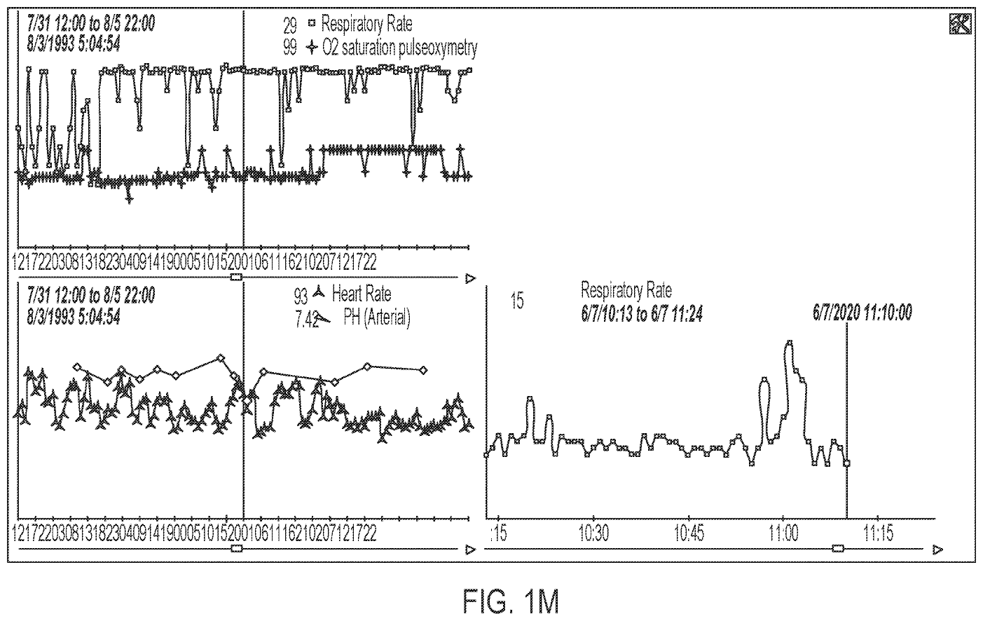

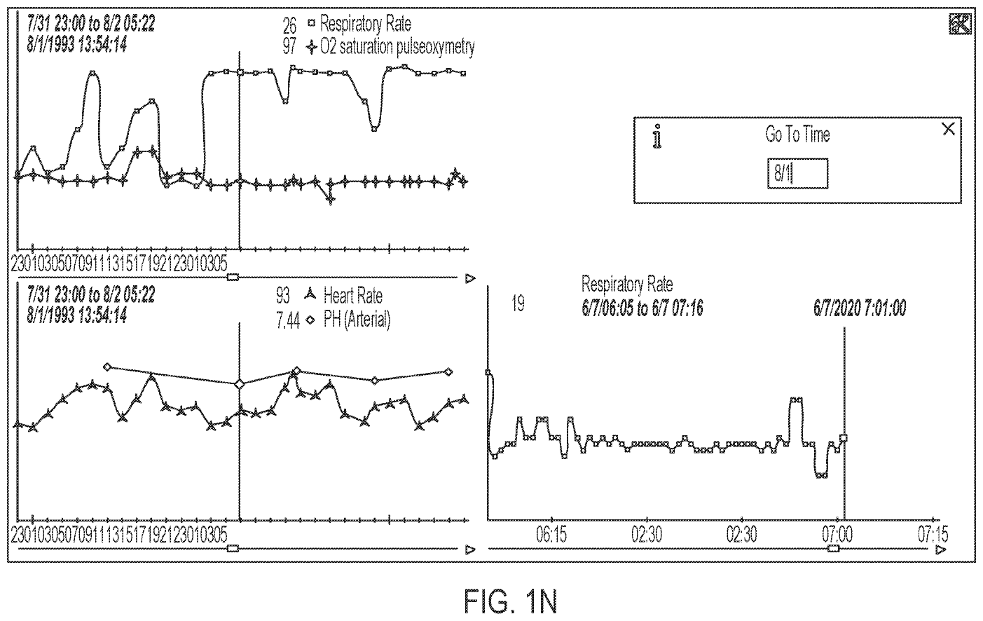

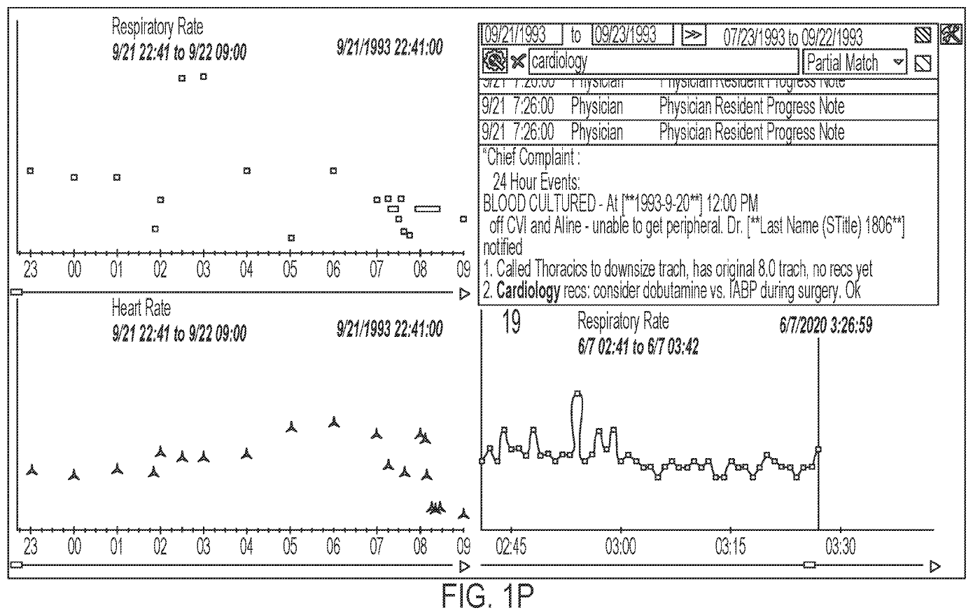

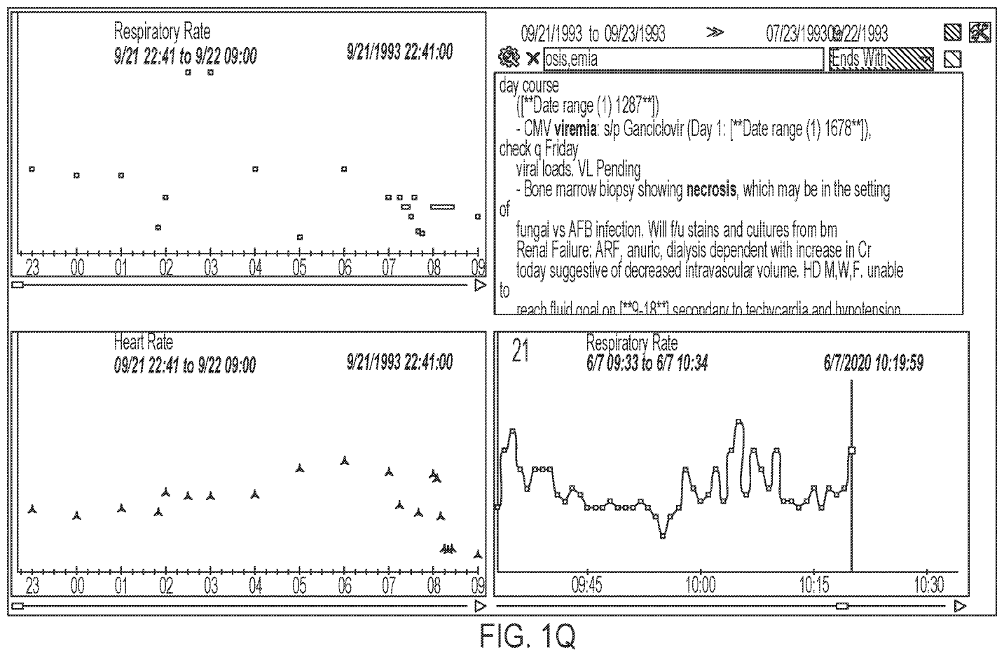

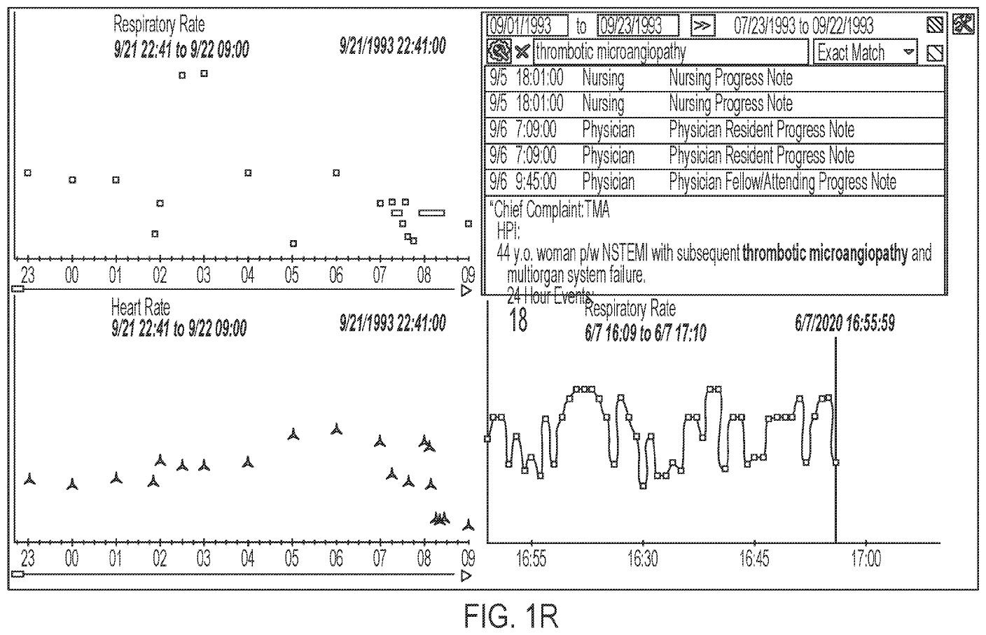

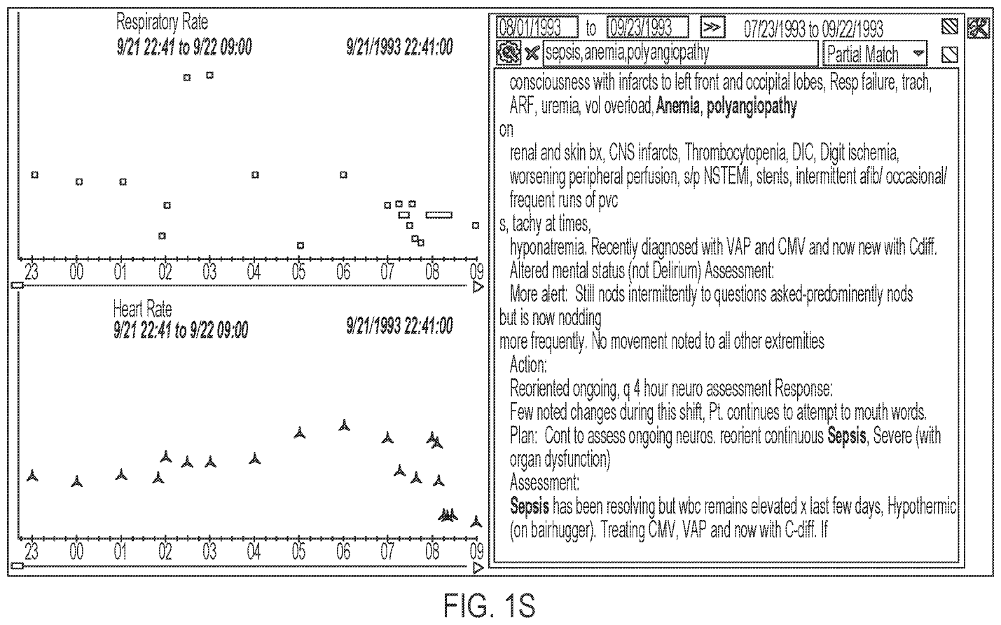

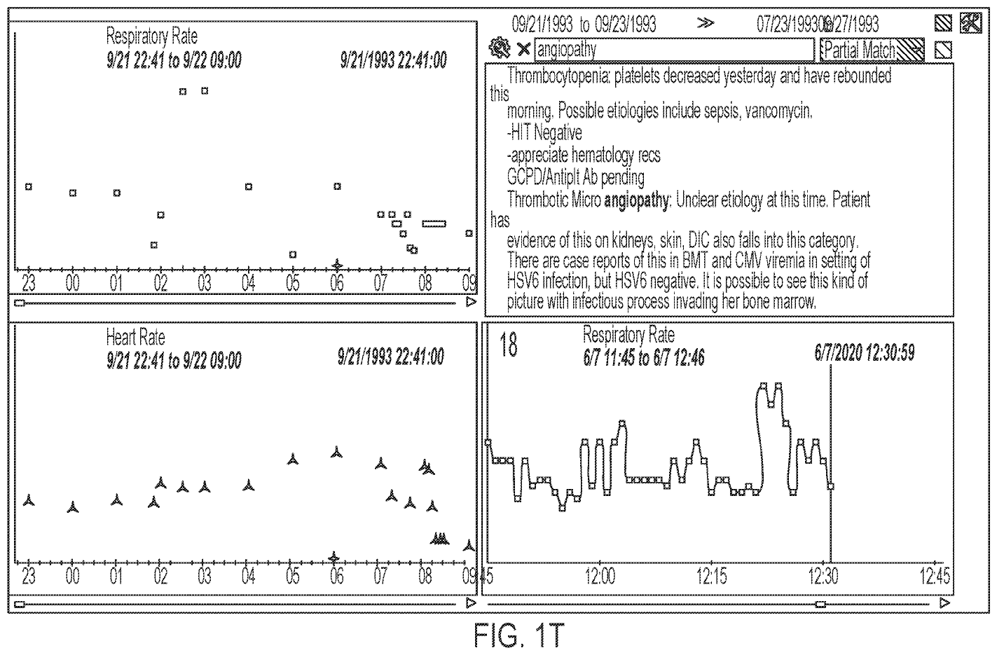

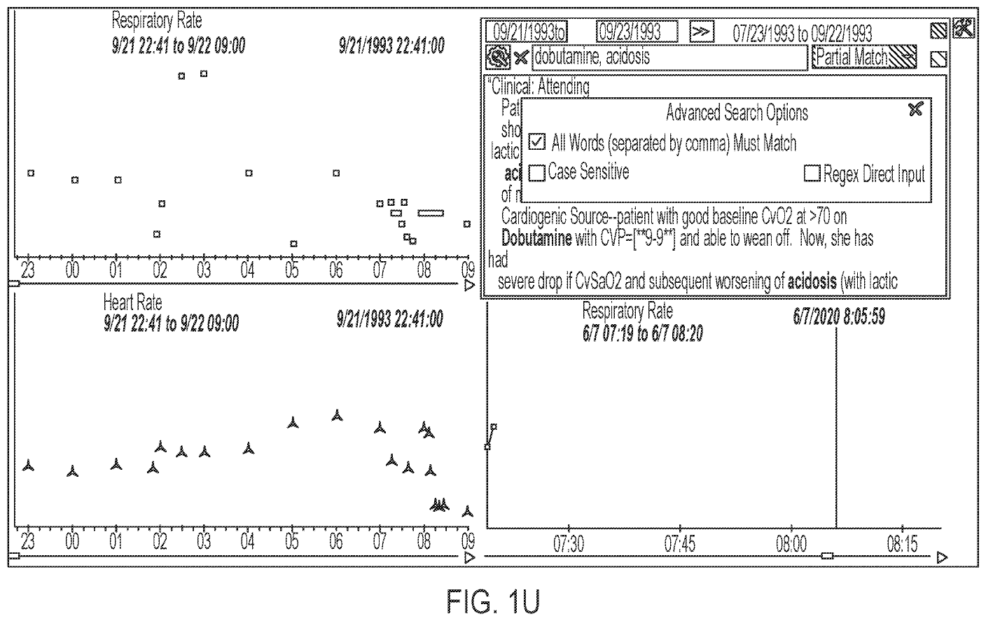

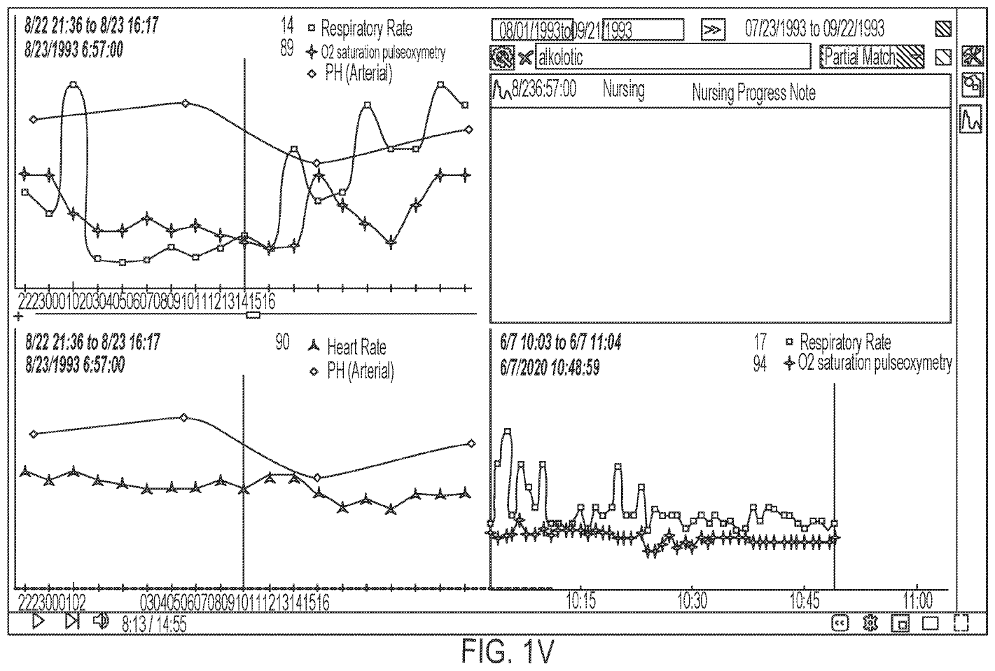

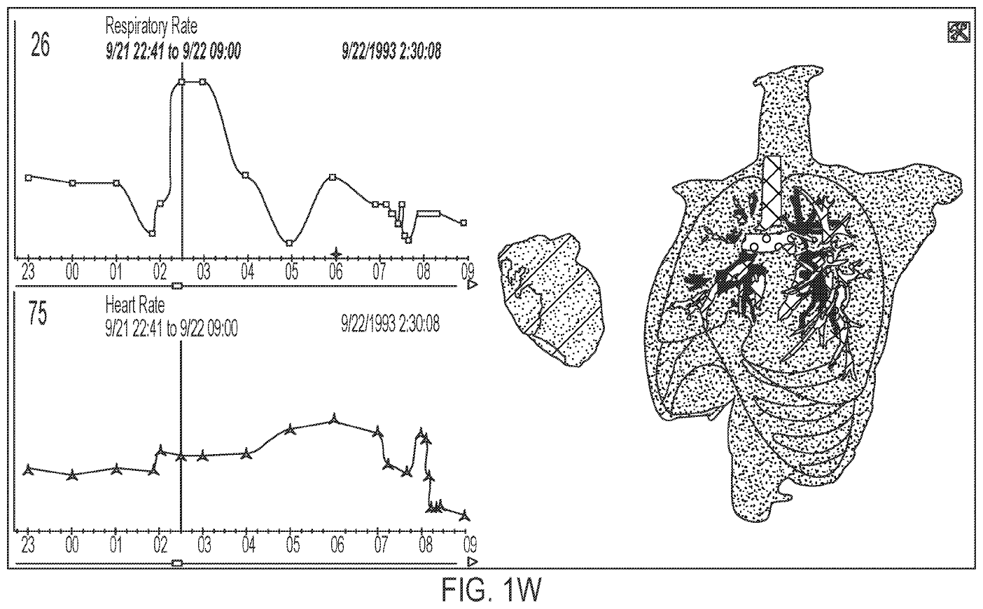

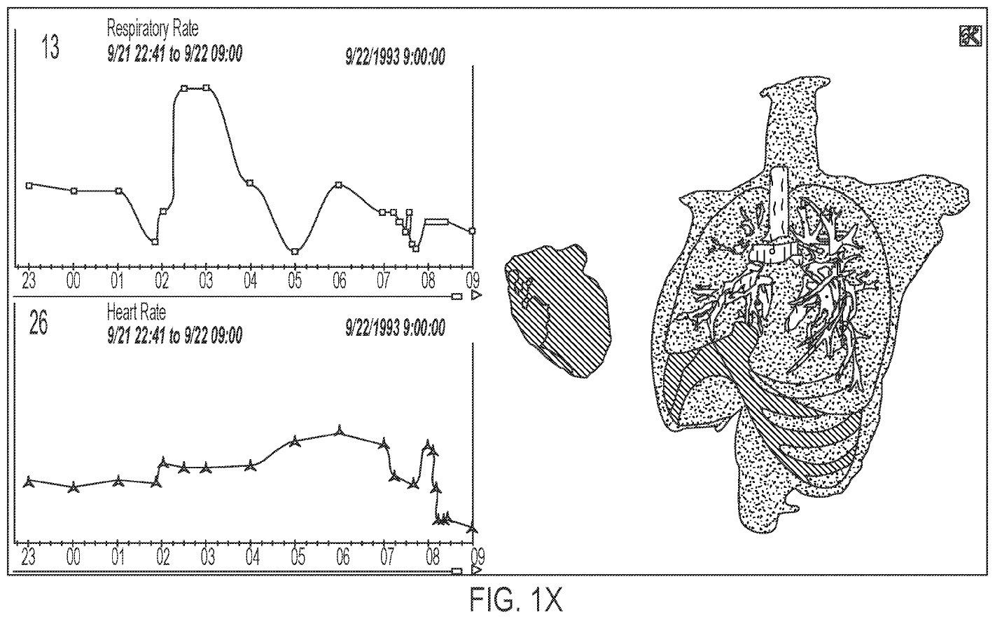

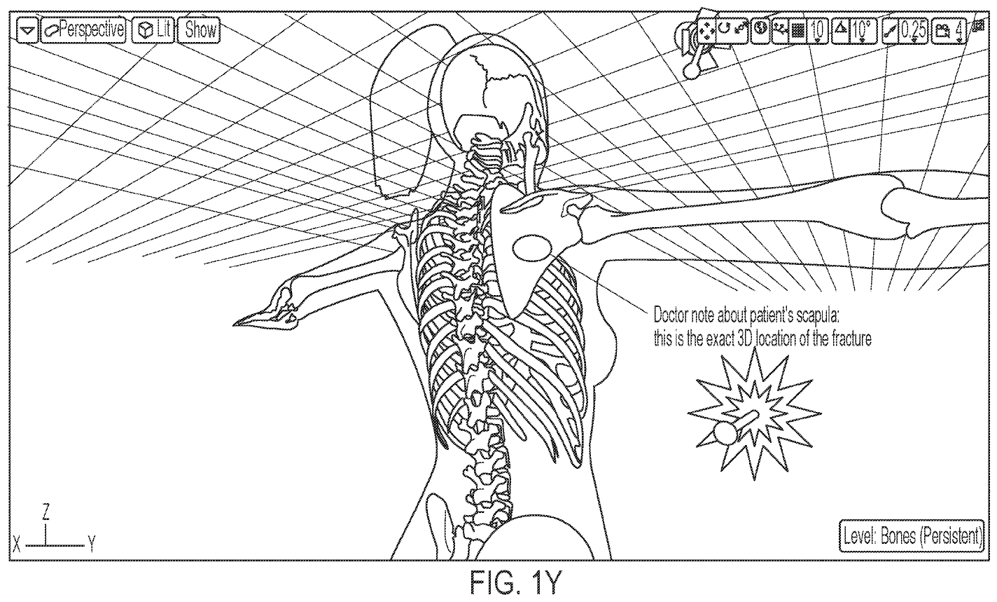

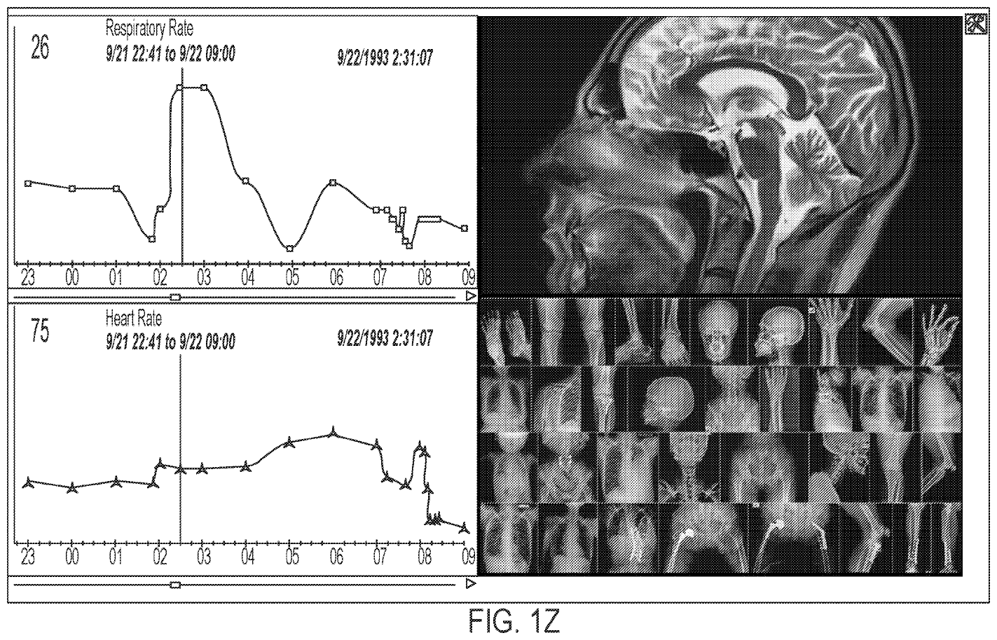

FIG. 1 illustrates a block diagram of an example system 100 for remotely monitoring patients in an intensive care unit (ICU) environment, including managing and controlling the process of weaning patients from ventilators. The system 100 includes one or more medical devices 102 positioned within an ICU environment 103 and configured to capture biometric data associated with patients in the ICU environment. For instance, the medical devices 102 may include ventilators, arterial catheters, external pressure cuffs, pulse oximeters, electrocardiograms, pulmonary artery (PA) catheters, other bedside monitors, and any other suitable devices configured to capture biometric data associated with patients. Additionally, healthcare professionals may input certain types of biometric data associated with the patient as well. For instance, biometric data associated with patients may include one or more of: admission weight, admit type, age, blood pressure (e.g., arterial diastolic, arterial mean, arterial systolic), arterial CO2 pressure, arterial O2 pressure, creatinine, ethnicity, gender, Glasgow coma scale, height, hematocrit, inspired O2 fraction, mean airway pressure, O2 flow, oxygen saturation of blood, oxygen saturation pulseoxymetry, peak inspiration pressure, PEEP set, PH (arterial), plateau pressure, potassium, sodium, tidal volume, urine output, ventilator mode, white blood cell count, heart rate, respiratory rate, central venous pressure, right atrial pressure, PA pressure, cardiac output, and any other captured or recorded biometric data associated with patients in the ICU environment. The system 100 may store current and/or historical biometric data captured by the medical devices in a database 123.

The system 100 may further include one or more remote display devices 104 configured to graphically display indications of the health of the patients based on data associated with the patients captured by the medical devices 102. Finally, the example system 100 may include a computing device 106. The medical devices 102 and/or the remote display devices 104 may communicate with the computing device 106 via a network 108. Additionally, the medical devices 102, remote display devices 104, and/or the computing device 106 may further communicate with one or more healthcare computing device 110 via the network 108.

Each of the remote display devices 104 may include a user interface 111 configured to present information to users and/or receive various inputs from users. That is, each remote display device 104 may be configured to graphically display indications of the health of the patients based on the biometric data associated with the patients captured by the medical devices 102 via a respective user interface 111. In some examples, each remote display device 104 may be configured to display graphically display indications of the health of a particular patient (i.e., based on data captured by a particular medical device 102 or particular set of medical devices 102), while in other examples, each remote display device 104 may be configured to graphically display indications of the health of multiple patients, based on data captured by multiple medical devices 102 associated with various patients. In some examples, one or more remote display devices 104 may be positioned directly outside of rooms associated with particular patients, and each remote display device may be configured to display data associated with each respective patient. Additionally, in some examples, the remote display devices 104 may be positioned together in a room designed for patient monitoring by healthcare professionals. Moreover, in some examples, the remote display devices 104 may include mobile devices, such as laptops, smart watches, smart phones, tablets, etc., i.e., so that healthcare professionals may monitor patients from any location. In any case, each remote display device 104 may be positioned outside of the ICU environment 103 so that healthcare professionals can monitor patients without being exposed to diseases that may be present in the ICU environment 103. In some examples, the remote display devices 104 may be password protected, or may be authenticated by registered users using a security card 112. For instance, a remote display device 104 for a particular patient may be configured to remain in a locked mode unless a security card 112 associated with a healthcare professional for the patient is used to access the remote display device 104.

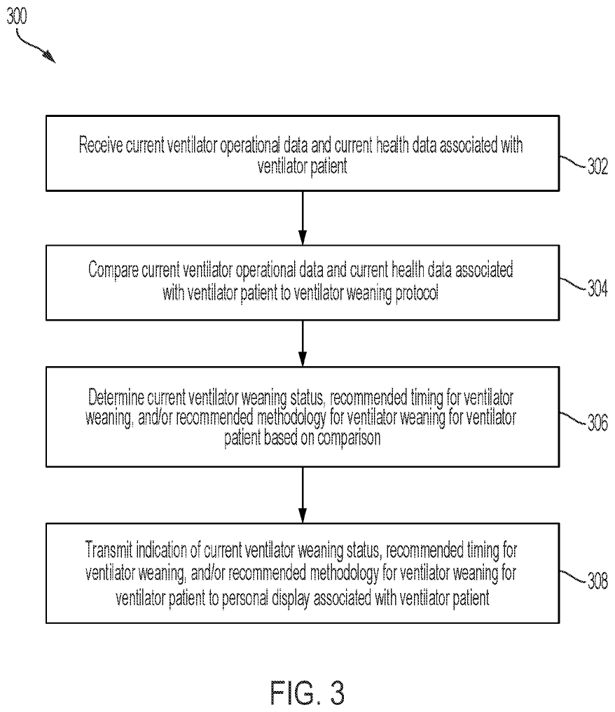

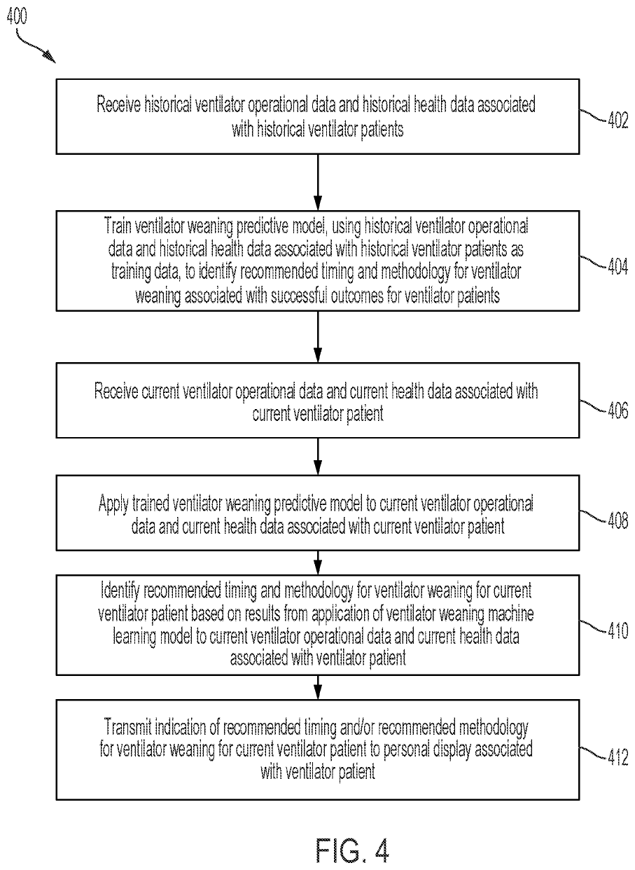

The computing device 106 may include a processor 114, such as one or more microprocessors, controllers, and/or any other suitable type of processor, and a memory 116 (e.g., volatile memory or non-volatile memory) accessible by the one or more processors 114 (e.g., via a memory controller). The one or more processors 114 may interact with the memory 116 to obtain, for example, computer-readable instructions stored in the memory 114 for executing a graphical display application 117, a control application 118, a ventilator weaning predictive model 120 and/or a ventilator weaning predictive model training application 122. In particular, the computer-readable instructions stored on the memory 114 may include instructions for carrying out any of the steps of the method 300 described in greater detail below with respect to FIG. 3 and/or the method 400 described in greater detail below with respect to FIG. 4.

The graphical display application 117 and the control application 118 may receive various data, including patient biometric data, other patient data (e.g., EMR data) and medical device settings data, including ventilator settings data, directly from the medical devices 102 or from the database 123 storing current and/or historical data captured by the medical devices 102, remote display devices 104, and/or healthcare computing device 110, and may analyze this data to generate graphics and/or information to be displayed via the remote display devices 104, i.e., via the user interfaces 111 of the remote display devices 104.

For example, FIGS. 1A-1H, 1J-1N, and 1P-1Z illustrate example user interface display screens that may be displayed via user interfaces 111 of remote display devices 104 used for remotely monitoring patients in an intensive care unit (ICU) environment, in accordance with some examples. For example, as shown in FIGS. 1A-1D, the user interface 111 may receive selections from a user of one or more types of biometric data to be displayed, and may accordingly display the selected types of biometric data over time in one or more graphs presented within a single screen by a user interface 111. For instance, as shown in FIGS. 1A-1D, the user interface 111 may receive selections from users regarding which types of biometric data are displayed in each graph, and may modify the display based on the selections. FIGS. 1E-1M illustrate various example graphs reflecting various types of biometric data over time within a single display screen.

In some examples, the graphical display application 117 may generate display items (e.g., graphs, three-dimensional animations, scalars, etc.) to be displayed by the user interfaces 111 of the remote display devices 104 that are color coded, generally on a two-sided spectrum—meaning that the “good” range will be in the middle as shown by green, and as the values go either above or below the normal range, the displays will move from green to yellow to orange to red. The graphical display application 117 may color code the display items according to a pre-defined protocol. In some examples, the graphical display application 117 may be configured to operate using various pre-defined protocols, and may receive a selection of one of the pre-defined protocols from a particular hospital or healthcare professional. Additionally, in some examples, the graphical display application 117 may define a new protocol based on selections from a user.

In particular, the graphical display application 117may normalize the biometric data such that each data point for each type of biometric data is sorted into one of several health status categories (e.g., a “normal” or “good” health status, several intermediate health statuses, a “bad” or “alarm” health status, etc.) based on a medical protocol. For instance, biometric heart rate data within a certain range of heart rates may be associated with a normal or good health status, while biometric heart rate data outside of that range may associated with an intermediate health status, and biometric heart rate data above or below certain threshold values may be associated with a bad or alarm health status. Accordingly, the graphical display application 117 may generate a single graph to display the various different types of normalized biometric data over time. For instance, the graphical display application 117 may generate a graph that includes a time axis and another axis that utilizes “lanes” for various health statuses (e.g., normal or good health status in a center lane, intermediate health statuses in bordering lanes above and below the center lane, and bad or alarm health statuses in highest and lowest lanes). Moreover, the graphical display application 117 may color-code the graphs based on the health status associated with each data point (e.g., with normal or good health status data points being shown in green, intermediate health status data points being shown in yellow or orange, bad or alarm health status data points being shown in red, etc.).

As shown in FIG. 1N, the user interface 111 may receive a selection by a user of a specific date and/or time, and may accordingly display graphs reflecting biometric data captured at the selected date or time (or within a certain range of the selected date or time). That is, in a “normal” mode of operation, the user interface 111 may be configured to display moving wave forms of selected streaming biometrics at a current time. For instance, the user interface 111 may display current values of the selected biometrics and wave forms for a period of time in the past. The user interface 111 may receive user selections of a time, as shown in FIG. 1N, and may modify the displayed time span accordingly. Additionally, in some examples, the user interface 111 may receive haptic user input, such as “pinching in” and “pinching out,” i.e., similar to a GPS or map screen, and may modify the displayed time span based on this user input, e.g., by zooming out or zooming in. Moreover, in a “retrospective” mode of operation, the user interface 111 may be configured to do an “instant replay,” in response to a user selection or command, by moving the vertical time line to any time slice in the past, and allowing it to go forward—either real time or a time-compressed fast-forward mode. Moreover, the user interface 111 may be configured to receive user input in the form of “swipe-left” or “swipe-right” input, and may modify the display to show different.

In some examples, as shown in FIG. 1P-1V, the user interface 111 may display searchable nurse's notes that allow custom search input and word-based analysis and text-mining beyond simple word look-ups. Moreover, in some examples, the user interface 111 may be configured to display a side toolbar displaying specific scalar values and indexes (e.g. BMI, diabetes indicators, etc.) to ensure that all co-morbidities are displayed. That is, as shown in FIG. 1P-1V, the graphical display application 117 may receive text notes as user input (e.g., from an attending healthcare professional, such as a doctor or a nurse, via the user interface 111) regarding the patient's health status at various times, and may store these notes including an indication of the time at which they are recorded. The graphical display application 117 may then subsequently receive user input including search terms, and may locate instances of the term in historical notes and times at which the historical notes were recorded. The graphical display application 117 may then cause the user interface 111 to display indications of times at which notes related to a given term were recorded, so that other users (or the same user) can easily search for terms within the notes and see an indication of a time at which notes related to a certain term were recorded. For instance, in some examples, the graphical display application 117 may store these notes in the database 123 along with the medical device data. The graphical display application 117 may cause these notes to be displayed simultaneously with the graphs discussed above on a single screen via the user interface 111 so that a user can see biometric data recorded at the time of any given note.

For example, when a user inputs a particular search term(e.g., “sepsis”) via the user interface 111, the graphical display application 117 may locate instances in which the term appears within the text notes, along with times associated with each instance, and may cause the user interface 111 to display the instances and associated times. For instance, the user interface 111 may display the text notes in which the term appears and may highlight the term within notes in which it appears, e.g., as shown at FIGS. 1P-1U. Moreover, the user interface 111 may receive a selection, from a user, of a particular instance where the term appears within the text notes, and the graphical display application 117 may cause the user interface 111 to display graphs of biometric data as recorded at the time (or within a range of times) associated with the selected instance of the term based on the selection, e.g., as shown at FIG. 1V. For instance, as shown in FIG. 1V, when the user interface 111 receives a selection, from a user, of a particular instance of the term “alkolotic” in a healthcare professional's text note from 6:57 AM on a particular day, 8/23, the graphical display application 117 may cause the user interface 111 to display one or more graphs of biometric data recorded at 6:57 AM on 8/23 (or from a range of times around 6:57 AM on 8/23, etc.) For example, as shown in FIG. 1V, the graphical display application 117 may cause the user interface to display a graph of the patient's respiratory rate, O2 saturation pulseoxymetry, pH (arterial), and heart rate at 6:57 AM on 8/23, when the selected term was noted. Moreover, the graphical display application 117 may cause the user interface to highlight or otherwise identify the exact time and date associated with the selected term within the graphs of the biometric data (e.g., using a vertical line as shown in FIG. 1V). Advantageously, these features help users to visually connect various noted conditions or observations to graphs displaying biometric data from times at which the conditions were noted. Additionally, these features allow for a smoother transfer of knowledge/information across healthcare professionals' shift changes, because a healthcare professional in a later shift can easily identify which patient conditions were noted by healthcare professionals in earlier shifts, and can the patient's biometric data from past times when these conditions were noted. Furthermore, these features allow healthcare professionals to easily identify patterns, trends, or anomalies in patient conditions and biometric data across multiple shift changes.

Moreover, in some examples, as shown in FIG. 1W and 1X, the graphical display application 117 may cause the user interface 111 to display three-dimensional models of a patient's organs, e.g., including the heart and lungs of the patient, alongside the one or more graphs as discussed above with respect to FIGS. 1A-1H and 1J-1N. For instance, the graphical display application 117 may generate the three-dimensional models of the patient's organs such that the models, as displayed by the user interface 111, are dynamically animated to reflect biometric data associated with the patient. For instance, the graphical display application 117 may generate a three-dimensional model of the patient's heart that is animated to reflect the patient's actual heart rate based on biometric data captured by ICU medical devices (i.e., at a current time or at a past time selected by a user of the remote display device). Similarly, the graphical display application 117 may generate a three-dimensional model of the patient's lungs that are animated to reflect the patient's actual respiratory rate based on biometric data captured by ICU medical devices (i.e., at a current time or at a past time selected by a user of the remote display device). In some examples, the user interface 111 may display these three-dimensional models simultaneously with graphs reflecting the biometric data associated with the patient over time. That is, the graphical display application 117 may generate three-dimensional models that are animated to reflect a patient's breathing rate and heart rate relative to the actual biometric values present on the graphs. Additionally, the graphical display application 117 may color-code the three-dimensional models of the patient's body areas to match the normalized values of the raw biometric readings shown in the graphs. For instance, the graphical display application 117 may generate a three-dimensional animation of the patient's heart (or a relevant portion of the patient's heart) that is shown in green when normalized biometric data associated with the patient's heart (or associated with the relevant portion of the patient's heart) is in a “good” range, and shown in red when normalized biometric data associated with the patient's heart (or associated with the relevant portion of the patient's heart) is in a “bad” or “alarm” range. Accordingly, as displayed by the user interface 111, as the data shown in the graphs changes, the model animations and colors will continuously match the changing biometric values. In particular, if the user interface 111 receives input from the user adjusting a graph reflecting the patient's biometric data over time to select a certain time or a certain time period (e.g., a one-hour period five hours ago), the graphical display application 117 may animate the three-dimensional models of the user's body areas may displayed by the user interface 111 to reflect the patient's biometric data as captured at the selected time or over the selected time period.

Furthermore, in some examples, as shown in FIG. 1Y, the user interface 111 may display a three-dimensional model note-taking view for use by healthcare professionals. That is, based on input from the user, the user interface 111 may dynamically update the three-dimensional display to move around a three-dimensional model of the patient's body. The user interface 111 may further receive user input indicating specific bones, muscles and organs, from the displayed three-dimensional model associated with issues related to the patient. That is, the user interface 111 may receive “tags” or otherwise notes (written and/or voice) associated with specific points within the three-dimensional model of the patient's body, and the graphical display application 117 may store the tags or notes in a database (e.g., database 123) so that other healthcare professionals associated with the patient can easily review all notes (written or voiced) associated with the patient's body in a three-dimensional view. In some examples, the graphical display application 117 may cause the user interface 111 to rotate the three-dimensional model of the patient's body along any axis and any angle based on input from the user, and may receive notes from the user based on user input selecting different body regions within the three-dimensional model of the patient's body. The graphical display application 117 may add these text-based notes to the database, including an indication of the tagged point on the patient's body, along with the perceived three-dimensional view of the creator of the note. Accordingly, the graphical display application 117 may cause the user interface 111 to display the tagged three-dimensional location in a three-dimensional immersive view when the user interface 111 receives a subsequent user's selection of the same note.

Additionally, in some examples, as shown in FIG. 1Z, the user interface 111 may display radiology imaging (i.e., in addition to graphs and/or three-dimensional models of the patient's body). Various images may be captured from various radiology imaging devices (e.g., X-Ray, sonogram, CT scan, etc.). For instance, a hospital's radiology department may send these radiology images to an online database (e.g., database 123) accessible by the remote display device, which can show multiple radiology images simultaneously, including in a grid, for maximum understanding of the radiology results. In some examples, the graphical display application 117 may cause the user interface 111 to display a three-dimensional animated “tour” of image-series data (e.g. a CT Scan or PET scan). Moreover, the user interface 111 may receive “tags” or other to tag written or voiced notes for specific regions within the image-series data (i.e., in a similar manner as discussed above with respect to the three-dimensional model of the patient's body). The graphical display application 117 may cause the user interface 111 to display a specific region within the image-series data associated with a particular tag or note when the user interface 111 receives a selection of the particular tag or note from a subsequent user.

Furthermore, in some examples, the control application 118 may generate the graphics and/or information based on comparing the data to a ventilator weaning protocol (e.g., a ventilator weaning protocol retrieved from a ventilator weaning protocol database 124). The control application 118 may transmit the generated graphics and/or information to the remote display devices 104 (or to healthcare computing devices 110) via the network 108.

For example, the control application 118 may generate indications of each patient's ventilator settings as well as a visual system (red/yellow/green background) highlighting each patient's trajectory on the ventilator as they progress in the ventilator weaning process for display via remote display devices 104 positioned outside of each ventilator patient's room. That is, generally speaking, ventilator weaning may be dived into three phases: (1) preparing to wean, (2) weaning and (3) extubation, and the remote display device may display a patient's current phase, as well as a status associated with that phase. For example, if a patient is weaning and their SpO2 is declining, the screen may display a ‘W’ with a red background. As another example, if a patient is preparing to wean, but making no progress, the screen may display a ‘P’ with a yellow background. Additionally, as another example, if a patient is ready to extubate, the screen may display an ‘E’ with a green background. Similarly, as another example, if a patient is successfully weaning, the screen may display a ‘W’ with a green background. Furthermore, as another example, if a patient is fully prepared to begin weaning, the screen may display a ‘F’ with a green background.

Moreover, in some examples, based on the based on comparing the various data (e.g., patient data, ventilator settings data, etc.) to a ventilator weaning protocol (e.g., a ventilator weaning protocol stored in the ventilator weaning protocol database 124), the control application 118 may generate indications of clinical guidance, e.g., to recommend/remind a treating healthcare professional (or team of healthcare professionals) of the need (when appropriate) for a spontaneous breathing trial (SBT) for a particular ventilator patient or other recommendations (e.g., a recommendation for extubation, a recommendation to modify ventilator settings, or various other recommendations related to ventilator weaning) and may transmit this generated indication to a remote display device 104 positioned outside of the room of the ventilator patient (or to a healthcare computing device 110 associated with the patient's healthcare provider). The remote display device 104 or the healthcare computing device may then display the generated indication. Furthermore, in some examples, the control application 118 may generate the graphics and/or information and/or indications of clinical guidance discussed above based on applying a predictive model 120 to the data.

For example, the predictive model 120 may be trained using a predictive model training application 122. In some examples, the predictive model training application 122 may train a machine learning model (e.g., using Kalman filtering, support vector machine, logistic regression and boosting, etc.) to identify metrics such as readiness to wean, weaning parameters, time of extubation, factors associated with successful extubation, etc., e.g., using information stored in a historical ventilator patient database 126 and/or information stored in a historical ventilator operational database 128 as training data. For example, the historical ventilator patient database 126 and historical ventilator operational database 128 (or various other databases accessed by the computing device 106 but not shown in FIG. 1) may store clinical data associated with mechanically ventilated ICU patients.

In some examples, the predictive model training application 122 may train the predictive model 120 using machine learning techniques, while in other examples the predictive model training application 122 may train the predictive model 120 using a multiple hypothesis testing approach, or a mix of machine leaning approaches and multiple hypothesis approaches. That is, some variables associated with ventilator weaning are correlated and some of them are uncorrelated. In some examples, multiple hypothesis testing approaches may be used to determine the most accurate predictor for those variables and to evaluate the significance level of each. Advantageously, multiple hypothesis testing approaches treat type-I and type-II error separately, while most other approaches fail to make a difference when dealing with both.

The predictive model 120 may be trained (using the predictive model training application 122) to select the best indicators for weaning and to predict those weaning indices. The predictive model 120 may use real time forecasting approaches to predict the parameters during the day, providing an advantage over current methods in which the evaluating the readiness of patients is performed only once a day. The predictive model training application 122 may train the predictive model 120to identify the best time for weaning from a ventilator during the day when dealing with the multi-scale time-series of different parameters. Additionally, the predictive model training application 122 may train the predictive model 120 by testing the optimal time to wean as well as the rate of chronic weaning can also be tested using multiple hypothesis testing approaches.

Hundreds of variables may be analyzed, including patient factors (SpO2>92%, tapering/low doses of vasopressors, vital signs acceptable [e.g., BP>=90 systolic, HR=55 to 135 bpm] age, weight, race, sex, surgical, mortality, PIM3, heart rate, etc.), ventilator factors (FiO2<=50%, PEEP<=5-8 cm H2O, hemodynamic stability, oxygenation and ventilation, PETCO2, f total, breath/min, etc.), ABG Parameters (PaO2>=75 mmHg, pH>7.25, etc.), other factors (gas exchange, breathing pattern, FVC>15 ml/kg, CROP index, IWI, CORE, P0.1/PImax>0.3, Mechanical ventilation score, Ventilation AV and Breathing frequency goal, Oxygenation VILI, AO, LRP, LRVT, PIP, PEEP, ICU LOS, Hospital LOSetc.), etc.

Furthermore, the predictive model training application 122 may use multiple hypothesis testing approaches to select the top significant variables may be selected while controlling the type-I error at 5%, with a confidence level for each variable. Additionally, the predictive model training application 122 may use machine learning approaches to select variables that are most important for prediction accuracy. Additionally, the predictive model training application 122 may use feature engineering in artificial intelligence to create the significant variables based on the given information. Accordingly, after this selection process, there will be some number (n) of selected variables (e.g., V₁, V₂, V₃, . . . , Vₙ).

While machine learning models provide great accuracy of predicting the readiness to wean and different features/variables, in practice, different types of error have different importance. That is, in practice, the false positives and false negatives are of different importance; but most machine learning approaches fails to handle the difference. However, using multiple hypothesis testing, it is possible to provide the optimal solution. In particular, multiple hypothesis testing approaches may be used to determine type I error and type II error separately, and with certain error boundaries for both, or with certain optimal weight for both, in order to obtain an optimal solution for patients.

Moreover, in some examples, due to anticipated data imbalances, the predictive model training application 122 may apply several different data balancing processes, followed by machine learning and real time forecasting approaches (e.g., Kalman filtering, support vector machine, logistic regression and boosting). Furthermore, the predictive model training application 122 may train and test the predictive model 120 on different balanced datasets. Reduction of Type I & II errors may be accomplished via multivariate hypothesis techniques. Additionally, the predictive model training application 122 may refine the predictive model 120 as new data is collected by ventilators (i.e., by ventilators that are part of the medical devices 102), remote display devices 104, and/or healthcare computing device 110 and added to databases 126 and 128.

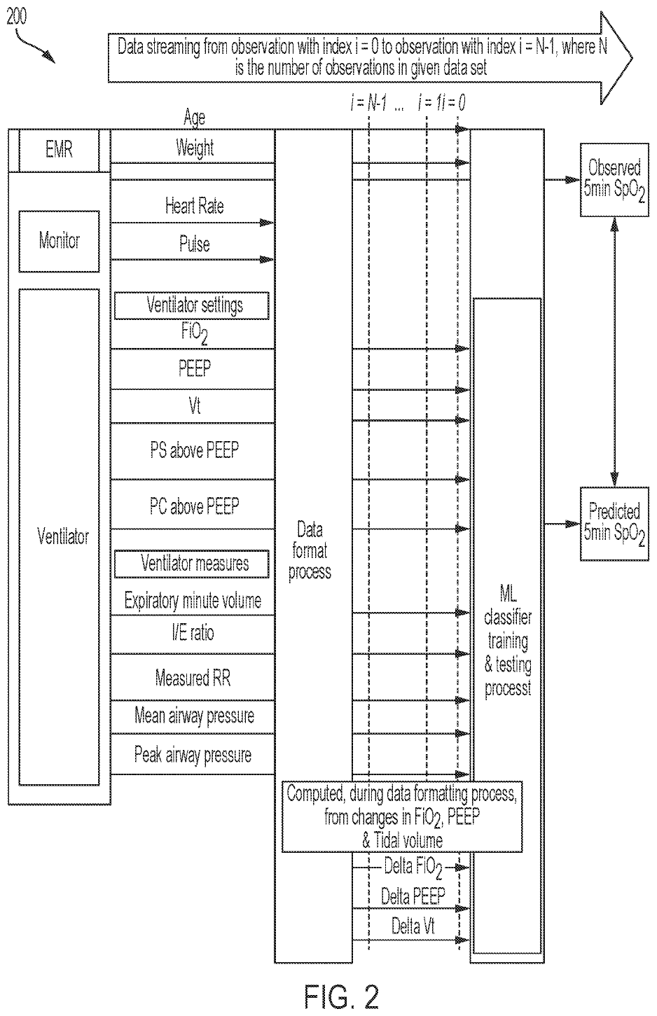

The success criteria for prediction is the ability of the predictive model 120 to predict the weaning outcomes based on the EMR and ventilator settings (i.e., delta in inspired fraction of Oxygen (ΔFiO2), delta in tidal volume, pressure support or pressure controlled (ΔVt, ΔPS or ΔPC) or delta in positive end expiratory pressure set (ΔPEEP)). Example variables used in the model are detailed in FIG. 2 within their sources, means of extraction and a schematic of the main components of the study.

In some examples, the predictive model training application 122 transforms data from the linear format into a table, where the clinical variables are the column labels and the patient codes are stored as the row labels. Since the readings for the various variables involved are not all set at the same frequency, the data for the different variables will require alignment along the rows time-steps. Then, only the rows at which at least one of the setting variables is modified will be preserved in the data file. Binning of the target variable data into several classes (e.g., scoring for readiness to wean) allows for better classification performance. For all time-steps, key values will be validated and kept in the database if other metrics from monitors in each row are within 10% of other sensors measuring the same data points. In some cases, rows containing readings which do not fit this condition will be removed. The number of rows will be randomly distributed between the training and test databases, without considering the number of patients in each dataset.

In any case, the control application 118 may apply the trained predictive model 120 to a particular patient's current patient data and/or current ventilator settings (e.g., obtained from ventilators that are part of the medical devices 102, remote display devices 104, healthcare computing device 106, etc.) in order to identify parameters with a high likelihood of leading to successful extubation for that particular patient. The control application 118 may then use the identified parameters to generate graphics and/or information, including indications of the patient's trajectory on the ventilator and/or indications of clinical guidance or other recommendations for the patient, and transmit the generated graphics and/or information to the remote display device 104 associated with that patient (or to a healthcare computing device 110 associated with the patient's healthcare provider).

FIG. 3 illustrates a flow diagram of an exemplary method 300 for managing and controlling the process of weaning a patient from a ventilator based on a ventilator weaning protocol (e.g., the ventilator weaning protocol from the database 124 described above), in accordance with some embodiments. One or more steps of the method 300 may be implemented as a set of instructions stored on a computer-readable memory 116 and executable on one or more processors 114.

As shown in FIG. 3, current ventilator operational data and current health data associated with a ventilator patient may be received or accessed (block 302). The current ventilator operational data and current health data associated with the ventilator patient may be compared (block 304) to a ventilator weaning protocol. The patient's current ventilator weaning status, a recommended timeline for ventilator weaning for the patient, and/or a recommended methodology for ventilator weaning for the patient may be determined (block 306) based on the comparison. An indication of the patient's current ventilator weaning status, a recommended timeline for ventilator weaning for the patient, and/or a recommended methodology for ventilator weaning for the patient may be transmitted (block 308) to a remote display device associated with the ventilator patient.

FIG. 4 illustrates a flow diagram of an exemplary method 400 for managing and controlling the process of weaning a patient from a ventilator based on a predictive model for ventilator weaning (e.g., the predictive model 120 described above), in accordance with some embodiments. One or more steps of the method 400 may be implemented as a set of instructions stored on a computer-readable memory 116 and executable on one or more processors 114.

As shown in FIG. 4, historical ventilator operational data and historical health data associated with a plurality of historical ventilator patients may be received or accessed (block 402). A ventilator weaning predictive model may be trained (block 404) to identify recommended timing and methodology for ventilator weaning associated with successful outcomes for ventilator patients using the historical ventilator operational data and historical health data. Current ventilator operational data and current health data associated with a ventilator patient may be received or accessed (block 406). The trained predictive model may be applied (block 408) to the current ventilator operational data and current health data associated with the ventilator patient. Based on the results of applying the trained predictive model to the current ventilator operational data and current health data associated with the ventilator patient, a recommending timing and/or methodology for ventilator weaning for the patient may be identified (block 410). An indication of a recommended timeline for ventilator weaning for the patient, and/or a recommended methodology for ventilator weaning for the patient may be transmitted (block 412) to a remote display device associated with the ventilator patient.

1. A system for remotely monitoring hospital patients, the system comprising:
one or more medical devices configured to capture one or more types of biometric data associated with a patient, the medical devices and the patient being positioned in an intensive care unit (ICU) environment; and
a remote display device positioned external to the ICU environment and configured to display, via a user interface, an indication of the health of the patient based on the captured biometric data, the indication including a graph mapping one or more normalized health statuses associated with the patient over a period of time, wherein each normalized health status is based on normalizing a particular type of the captured biometric data based on a medical protocol, such that each data point of the graph includes an indication of a normalized health status associated with the patient at a given time.

2. The system of claim 1, wherein the indication of the health of the patient further includes an animated three-dimensional model associated with the body of the patient, the animated three-dimensional model being dynamically updated to reflect biometric data associated with the patient captured over the period of time.

3. The system of claim 1, wherein the one or more medical devices include one or more of: ventilators, arterial catheters, external pressure cuffs, pulse oximeters, electrocardiograms, and pulmonary artery (PA) catheters.

4. The system of claim 1, wherein the one or more types of biometric data include one or more of: blood pressure, oxygen saturation of blood, heart rate, respiratory rate, central venous pressure, right atrial pressure, PA pressure, and cardiac output.

5. The system of claim 1, wherein each of the normalized health statuses is one or more of: a normal health status, one or more intermediate health statuses, and an alarm health status.

6. The system of claim 1, wherein each normalized health status is associated with a color as shown in the graph.

7. The system of claim 1, wherein the medical protocol is selected by a user from a number of available medical protocols.

8. The system of claim 1, wherein the medical protocol is created by a user.

9. The system of claim 1, wherein the medical protocol includes a range of values for each type of biometric data associated that are associated with each normalized health status.

10. The system of claim 1, wherein the one or more types of biometric data upon which the normalized health statuses shown in the graph are based are selected by a user.

11. The system of claim 1, wherein the period of time is selected by a user.

12. The system of claim 1, wherein the remote display device is further configured to:
receive one or more text notes associated with the patient as user input; and
store indications of times at which each of the one or more text notes were recorded.

13. The system of claim 12, wherein the remote display device is further configured to:
receive an indication of a term to be searched within the text notes as user input;
search the text notes for instances of the indicated term within the text notes; and
display any instances of the indicated term within the text notes.

14. The system of claim 13, wherein the period of time is selected based on a time at which a text note including an instance of the indicated term was recorded.

15.-43. (canceled)

44. A method for displaying an indication of health of a patient over time, the method comprising:
normalizing, by one or more processors, one or more types of biometric data associated with a patient based on a medical protocol to produce a normalized health status for each type of biometric data;
generating, by the one or more processors, an indication of the health of the patient based on the biometric data, the indication including a graph mapping one or more normalized health statuses associated with the patient over a period of time, such that each data point of the graph includes an indication of a normalized health status associated with the patient at a given time;
displaying, by the one or more processors, the generated indication of the health of the patient via a user interface display.

45. A method for displaying an indication of health of a patient over time, the method comprising:
receiving, by one or more processors, one or more types of biometric data associated with a patient captured over a period of time;
generating, by the one or more processors, an animated three-dimensional model associated with the body of the patient, wherein one or more features of the animated three-dimensional model associated with the body of the patient are dynamically updated as the one or more types of biometric data associated with the patient are change over the period of time; and
displaying, by the one or more processors, the generated animated three-dimensional model associated with the body of the patient via a user interface display.

46. The method of claim 45, further comprising:
receiving, by the one or more processors, an indication of a period of time selected by a user;
dynamically updating, by the one or more processors, the animated three-dimensional model associated with the body of the patient to reflect biometric data associated with the patient captured by the one or more medical devices over the period of time selected by the user.

47. A method for displaying an indication of health of a patient over time, the method comprising:
receiving, by one or more processors, one or more types of biometric data associated with a patient captured over a period of time;
receiving, by the one or more processors, one or more text notes associated with the patient entered by a user over the period of time;
storing, by the one or more processors, indications of times at which each of the one or more text notes were recorded;
receiving, by the one or more processors, an indication of a term to be searched within the one or more text notes associated with the patient entered by the user;
searching, by the one or more processors, the one or more text notes associated with the patient entered by the user for instances of the indicated term within the one or more text notes;
displaying, by the one or more processors, an instance of the indicated term within the text notes via a user interface display; and
displaying, by the one or more processors, a graphic based on the one or more types of biometric data associated with the patient captured at the time at which the text note containing the instance of the indicated term was entered.

48.-59. (canceled)
